(19) United States
(12) Patent Application Publication
(10) Pub. No.: US 2009/0291400 A1
(43) Pub. Date: Nov. 26, 2009

(54) Liquid fuel lamp and fragrance diffuser apparatus

(76) Inventor: Erik H. Levy, San Francisco, CA (US)

Correspondence Address: LARRY D. JOHNSON, P.O. BOX 470277, CELEBRATION, FL, 34747, US

(21) Appl. No.: 12/154,566

(22) Filed: May 23, 2008

(51) Int. Cl. F23D 3/00; A61L 9/03; F23D 3/24; F23D 3/08; F23D 3/18; F23D 3/28; F21S 13/00; F21V 37/00

(52) U.S. Cl. 431/120

(57) ABSTRACT

A liquid fuel lamp design is disclosed, consisting of a length of wick housed within a vertical wick tube equipped with an upwardly-extending handle, which wick tube assembly is seated resealably within a funnel-shaped fuel aperture extending upward from a low wide fuel reservoir, atop which is securely affixed a lamp chimney of such dimension as to assure a high degree of isolation of the lamp flame and exhaust vapors above 451 F degrees from non-vapor combustibles in the lamp environment. The invention relies on a novel liquid fuel lamp filling system, whereby the wick tube may be equipped with a bushing and cap to resealably close the fuel aperture, and is removed and replaced by its upwardly extended handle via the chimney's upper opening to allow refilling and maintenance. The wick tube and fuel reservoir are configured so as to efficiently burn liquid fuels, especially viscous fuels, and in particular vegetable oils as fuel. The inner base of the lamp chimney accommodates non-flammable liquid fragrances such that while the lamp is burning, heat from the flame volatizes the fragrance and disperses it upon the column of hot vapor rising from the lamp chimney. The entire device is highly spill-resistant, constructed durably, and equipped with articulations to accommodate lamp accessories. A device for conveniently attaching and removing such accessories is also disclosed.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates generally to illuminating lamps and candles, and more particularly to a wick-type oil-burning low-light illumination device capable of burning liquid fuels, in particular, viscous fuels such as vegetable oil, and of diffusing fragrance.

### BACKGROUND INFORMATION AND DISCUSSION OF RELATED ART

Various types of vegetable oil burning lamps, wherein the oil is burned on a wick, have previously been suggested. For example, Sobelson U.S. Pat. No. 3,183,688 is directed to a floating dish-shaped device; supporting an upright wick in a bottom recess. In this type device, vegetable oil was added to the dish in an area directly surrounding the wick. The device was to be floated on water or other liquid, with the top of the wick ignited to produce a candle-like flame.

Wick tubes are commonly used in liquid fuel lamps, and are typically constructed of glass, copper, or other like materials. Most such lamps burn fuels less viscous and more volatile than vegetable oils, e.g. kerosene and other petrochemical fuel stocks, and alcohol.

U.S. Pat. No. 4,134,718 to Kayfetz, et al. discloses an oil-burning illuminating device capable of burning household vegetable oil and producing a candle-like flame. It includes a heat-resistant upright glass tube within which a fibrous wick is positioned, extending a short distance out the top. For supporting the tube and wick in a generally flat-bottomed container, such as a small drinking glass, a foot or stand having horizontally-extending projections and an upright sleeve may be connected to the bottom of the tube by insertion of the tube end in the sleeve. When the assembled tube, wick and foot are placed in a container and vegetable oil is added, at least to the level of the bottom of the wick, the extending portion of the wick may be ignited to produce a candle-like flame. The container is preferably transparent so that light is radiated in all directions, and a special container may be provided for seating atop a light-transmitting rod, so that light from the flame illuminates the rod.

U.S. Pat. No. 4,206,500 to Neil describes improvements in self contained illuminating devices, generally of the type otherwise referred to as candles, but differing from ordinary candles in that a container is employed into which a combustible oil or a meltable, combustible substance, such as wax or tallow, is placed, such as by pouring the same in a melted state such that upon hardening, the material conforms to the shape of the container. In particular, the focus of that invention is on an improved flame sustaining wick device constructed primarily of a non-rigid, typically low combustibility material having a cellular structure which will permit a drawing action by the wick of the liquid oil or melted material up through the wick to the flame. The wick of that invention includes an internally disposed stiffener including a base portion sufficient to support the wick within the container and in an essentially vertical free-standing attitude either in the presence of or in the absence of material and regardless of whether said material is in the liquid or solid state.

U.S. Pat. No. 6,667,006 to Richards teaches an emanator and lamp for dispersing volatiles. Disclosed is an oil lamp disperser and an oil lamp that disperses a volatile material from a fuel and volatile material mixture while the oil lamp is burning. The volatile material is released from the surface of an emanator element as the fuel and volatile material mixture passes through the emanator element prior to reaching the flame of the lamp. Proper construction of the lamp allows the lamp to volatilize substantial amounts of the volatile material into the surrounding environment.

U.S. Pat. No. 6,857,869 to Sun discloses a refillable candle stand. A candle stand includes a container and a bowl supported by an inner periphery of the container and having a hollow column formed on top of the bowl. The hollow column has multiple pawls each having a bend formed on a free end of the pawl and extending toward a central axis of the hollow column so as to clamp a wick. Multiple through holes are defined through a base of the bowl, wherein a space is defined between a bottom face of the container and the base of the bowl for receiving liquefied wax.

The foregoing patents reflect the current state of the art of which the present inventor is aware. Reference to, and discussion of, these patents is intended to aid in discharging Applicant's acknowledged duty of candor in disclosing information that may be relevant to the examination of claims to the present invention. However, it is respectfully submitted that none of the above-indicated patents disclose, teach, suggest, show, or otherwise render obvious, either singly or when considered in combination, the invention described and claimed herein.

### SUMMARY OF THE INVENTION

The liquid fuel lamp and fragrance diffuser apparatus of the present invention provides a wick-type vegetable oil burning lamp for emitting a soft candle-like light in all directions. The lamp consists of a wick-tube assembly consisting of a wick and a vertical wick tube equipped with an upwardly-extended handle seated resealably (e.g. by means of a bushing fitted around it's circumference) positioned over the fuel reservoir within the neck of an upwardly open and preferably funnel-shaped fuel aperture atop a low wide fuel reservoir, which wick tube seals the fuel aperture thoroughly while burning, and which aperture is closed completely when a resealably fitted cap (e.g. screw-on with gasket) closes the wick tube. These elements are arranged concentrically such that the wick-tube cap seals the wick tube which then seals the fuel aperture. This arrangement allows three modes of operation, namely portable mode, operating mode, and maintenance mode.

The vegetable oil in the fuel reservoir is drawn upward by the wick within the wick tube by capillary action. This primes the wick for lighting passively after time has allowed it to soak (users may prime the wick actively for immediate use by adding several drops of fuel to the tip of the wick). The inside diameter of the tube and the size of the wick are chosen such that capillarity through the tube-encased wick is maximized. During operation, the hot wick tube vaporizes fuel in the reservoir and expels the fuel vapor out the top to feed the flame. In order to ensure the lamp will burn until all fuel in the fuel reservoir is exhausted, the wick tube assembly is configured so that vaporization of fuel occurs even at the bottom of the wick tube in the following way. The wick tube is composed of a material of high thermal conductivity and low specific heat, such as copper, that becomes very hot along its entire length when the flame atop is burning, such that vegetable oil at the very bottom of the wick tube is volatized and expelled upward to feed the flame so long as it burns, even while the fuel reservoir is all but empty. The vertical height of the wick tube is also constrained to this effect. Hereafter, this specification is referred to as “vertical wick height” or VWH. The VWH must be less than one and one half inches for a copper wick tube of three-eighths inch internal diameter, and one-eighth inch wall thickness. It is anticipated that further research will allow this constraint to be increased.

The oil may be ignited at the short length of the wick which extends out the top of the tube. Once ignited, the inventive lamp apparatus will continue to burn oil for many hours, until all fuel in the fuel reservoir is exhausted.

Wick maintenance such as trimming and replacement is easily accomplished by lifting the wick tube out of the fuel aperture by means of the upwardly extended handle. Then the old wick stub may be removed and a fresh length of wick inserted in the wick tube, using tweezers if desired, and the length of wick extending from the top adjusted to an optimum one-eighth to one-quarter inch above the upper rim of the wick tube. The wick tube assembly may then be lit, or not, and replaced within the fuel aperture by means of the wick tube handle. In order to maximize bum-time between replacements, a wick longer than the wick tube may be used, provided that the tip of the wick is refreshed periodically between uses.

In the preferred embodiment, for safety the aforementioned arrangement is augmented by an upwardly extending chimney affixed securely (detachably or not) atop the fuel reservoir and around the wick tube assembly, of such dimension and shape as to partially or completely isolate the flame from non-vapor combustibles impinging on the lamp. Also, the chimney is of such dimension as to admit the fingers of adult users into the chimney to access the wick advance control on the wick tube and maintenance.

The chimney is preferably of a transparent yet durable material such as borosilicate glass, equipped with constrictions at the base immediately above the fuel reservoir and near the top, immediately below the rim. These constrictions simultaneously enhance the durability the chimney and provide articulation points for accessory devices that may be used with the lamp. In addition, in the event the lamp is knocked onto its side, these constrictions create a sump within the chimney into which oil may drip from the wick.

In the preferred embodiment, the wick tube is equipped with a wick advance control knob and associated mechanism within the wick tube, so that the wick may be conveniently adjusted for trimming as well as for light and heat level, either by lifting the wick tube assembly out of the chimney, or in situ.

Finally, an auxiliary device is disclosed whereby such accessories may be with one hand mounted and removed from the lamp and placed upright over any flat surface so as not to scorch that surface.

The present invention provides a wick tube modified to better bum vegetable oil (and other such relatively viscous liquid and nonvolatile liquid fuels), by means of the following specifications: (a) making it of a material with a low specific heat so that it gets very hot, and which has a high thermal conductivity (e.g. copper, brass, and like materials), and (b) using an adequately thick tube-wall (e.g., on the order of 1/16th inch for copper tubing) to increase viable wick tube height—see below, and (c) by restricting it's height to less than 1½″ (one and one half inches) extending up from the bottom surface of the fuel reservoir.

These specifications permit the flame at the top of the wick tube to create (by thermal conduction) temperatures sufficient to vaporize the vegetable oil or other liquid fuel at the base of the wick tube as well as at the upper limit adjacent to the fuel soaked wick. This allows production of a steady stream of fuel vapor up the wick tube even as the lamp runs through the last of the fuel supply. The vertical wick height cannot be increased beyond a given value (for any given combination of wick tube material and wall thickness) without leaving a puddle of fuel after the flame burns out.

Greater vertical wick heights (VWH) are desirable to allow greater fuel reservoir volumes for any given diameter of fuel reservoir. Greater VWH may be achieved by increasing the thickness of the wick tube wall to reduce radiating surface area per unit volume, achieving thereby higher operating temperatures in general, and specifically at the bottom of the wick tube, so as to allow fuel vaporization to continue even as the invention runs through the end of its fuel. It is expected that further research will uncover new means to increase viable VWH of the invention and/or to allow the lamp to bum even higher viscosity oils.

The present invention thus provides a wick tube for use with a closed fuel reservoir by the addition of an upwardly extended handle, as well as an O-ring affixed so as to allow a capped wick tube to be vertically supported by, close, and seal the overhead fuel aperture of a closed fuel reservoir.

It is therefore an object of the present invention to provide a new and improved wick- type liquid fuel lamp, optimized for viscous liquid fuels such as vegetable oil, which is relatively simple in construction, versatile in application, durable, convenient, and very safe to use.

It is another object of the present invention to provide a new and improved wick-type vegetable oil lamp which may be used to volatize fragrant oils to scent the air.

A further object or feature of the present invention is a new and improved wick-type vegetable oil lamp which supports the convenient, one-handed use of a variety of accessory attachments.

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a side elevation cross-sectional view of a liquid fuel lamp and fragrance diffuser apparatus of this invention in operation, illuminated and diffusing fragrance;

FIG. 2 is a side elevation cross-sectional view of the lamp apparatus with the wick tube assembly removed from the lamp body;

FIG. 3 is a side elevation cross-sectional view of a wick tube assembly, equipped with a wick advance mechanism and a screw-on cap;

FIG. 4 is a side elevation cross-sectional view of the lamp apparatus of this invention as configured for transport with the wick tube cap in place on the wick tube assembly, and the chimney covered by a chimney cover; and

FIGS. 5A and 5B are top plan views of an auxiliary device that allows an accessory to be mounted on the lamp and removed with one hand, and which functions also as a stand for such an accessory.

### DETAILED DESCRIPTION OF THE INVENTION

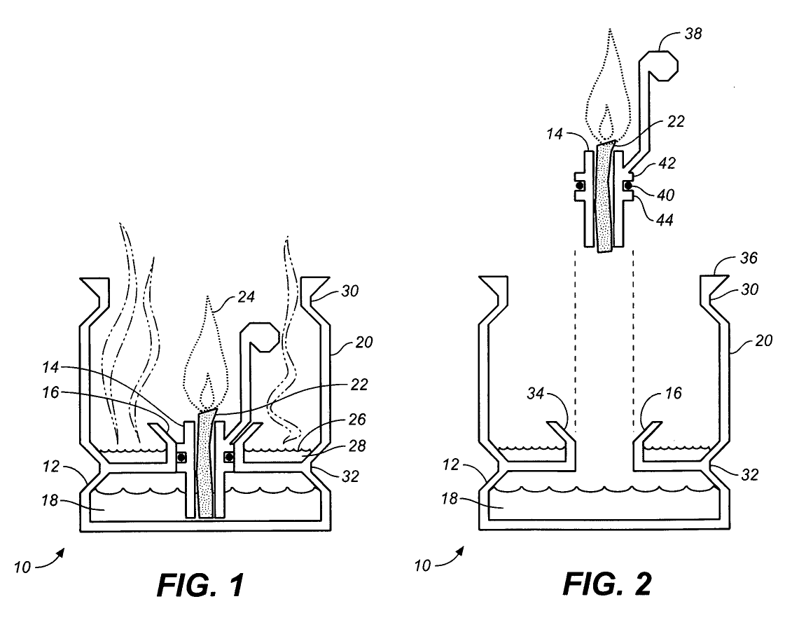

Referring to FIGS. 1 through 5B, wherein like reference numerals refer to like components in the various views, there is illustrated therein a new and improved liquid fuel lamp and fragrance diffuser apparatus of this invention, generally denominated 10 herein.

FIG. 1 is a side elevation cross-sectional view of a liquid fuel lamp and fragrance diffuser apparatus of this invention in operation, illuminated and diffusing fragrance. The apparatus includes lamp body 12, preferably constructed of a transparent, heat resistant material such as borosilicate glass (e.g. that sold under the trademark “Pyrex”), or alternately of opaque or translucent materials. Wick-tube assembly 14 is seated in place within the upwardly-extending fuel aperture 16 atop the fuel reservoir 18 within the lamp chimney 20. The chimney 20 may be perforated to allow passage of light, faster cooling, and for decoration, at the expense of durability and spill-resistance. The wick 22 is preferably natural fiber, and should be adjusted by the user to extend about one-fourth inch above the top of the tube in the wick tube assembly 14.

The inventive lamp apparatus operates to produce a controlled and isolated flame as follows: The user may light the lamp by withdrawing the wick tube or in situ within the chimney. Any source with a temperature in excess of the flashpoint of the fuel may be used to ignite the wick 22 soaked with fuel from the fuel reservoir 18. The heat of the flame vaporizes the fuel immediately below, which is replaced by fuel rising up the wick by capillary action along the fibers of the wick and the walls of the wick tube 14. The flame 24 heats the top of the wick tube 14, which progressively heats down it's length to its lower extent at the bottom of the fuel reservoir 18, such that the temperature at that point is maintained above the boiling point of the fuel during operation, so that fuel continues to vaporize until the fuel reservoir 18 empties completely.

The flame 24 is safely contained within the walls of the chimney, which dissipates the heat quickly by merit of the high thermal conductivity of its material and/or the greater radiating surface area per unit volume provided by perforation or other elaboration of the chimney. These measures minimize the chance of scalding the fingers of users during operation, and especially when a user picks up the invention after it has been overturned and yet burning for some time.

The inventive apparatus may be used to diffuse fragrance as follows: The flame 24 heats and volatizes the fragrance 26 placed within a fragrance reservoir 28 at the base of the chimney 20 and the heat flow out the top of the chimney diffuses the scent into the environment as shown. Only relatively non-flammable fragrances should be used, such as scented oil.

The lamp assembly 10 may be equipped a variety of useful attachments that articulate with the lamp at the upper constriction 30 or the lower constriction 32, or both, on the lamp body.

FIG. 2 is a side elevation cross-sectional view of the lamp apparatus with the wick tube assembly removed from the lamp body. The fuel reservoir 18 is accessed by fuel aperture 16 which may have on top an upwardly extending fuel funnel 34. The fuel reservoir 18 and fuel aperture 16 may be configured to accommodate the very short wick tube necessary to burn viscous liquid fuels like vegetable oil. For the sake of user-friendliness, the roof of the closed fuel reservoir should be sloped slightly upward toward the fuel aperture, so that when the lamp is turned upside down the contents will drain out of the reservoir. This allows the user to drain the reservoir conveniently, and also allows the reservoir to clear of water vapor more quickly after washing.

Securely affixed to the fuel reservoir 18 is a chimney 20 of a heat-resistant and durable, preferably transparent material such as borosilicate glass. The securely affixed chimney is an optional but important safety feature that substantially reduces the risk profile of the invention by isolating the flame from non-vapor flammables in the environment in all directions but the vertical. With regard to this remaining dimension of fire risk, the chimney 20 is high enough, alone or in combination with an accessory lampshade, that the vertical hot air stream from the flame falls below 451 degrees Fahrenheit (the flashpoint of cellulose, e.g. paper and wood) by adiabatic cooling before it rises out of the chimney/lampshade. The lampshade accessory may be securely affixed to the chimney utilizing the articulations available at the upper constriction 30 and lower constriction 32.

The inside diameter of the upper opening of the chimney 20 should be greater than 1½″ (one and one half inches) to permit finger access within the chimney. Likewise, the upper edge 36 should be at least 1½″ (one and one half inches) above the tip of the wick so that at a minimum the visible flame is horizontally contained below that level during proper operation.

This configuration provides for an unusually safe liquid fuel lamp, assuring that even if the lamp is tipped-over or inverted, the flame remains isolated within the heat-diffusing and durable, securely affixed chimney 20 enclosure. Morever this configuration is highly spill resistant: when the lamp is on its side, any fuel that may drip from the wick tube is captured within the chimney 20 in the sump formed between the upper constriction 30 and lower constriction 32. These constrictions also enhance the shock-resistance of the lamp. The chimney is preferably reinforced around it's upper edge 36 for durability.

Wick tube assembly 14 preferably includes an upwardly extended handle 38, an O-ring or other bushing 40 fitted to securely and resealably close and seal the fuel aperture 16, an upper ridge 42 and a lower ridge 44 to retain the bushing securely in place, and a wick 22 which should extend about a quarter inch above the upper edge of the wick tube and from there extend downward at least to the fuel reservoir floor and/or the base of the wick tube.

The handle 38, affixed to the wick tube 14 and upwardly extended is necessary if the lamp 10 is to conveniently accommodate a securely affixed chimney 20. The handle 38 allows the wick tube to be lifted clear of the lamp from within the chimney, even while burning, for convenient mess-free refilling of the fuel reservoir, and for maintenance such as wick trimming.

The handle 38 is nestled beneath the overhang of the upper constriction 30 of the chimney 20. This improves convenience of operation, allowing a quick-release action such that when the user simply pulls up from beneath the handle with a finger, the seal between the bushing 40 and fuel funnel 34 is broken and the wick tube swings free for removal, simultaneously exposing the fuel reservoir for refill and maintenance. This configuration also improves portability by reducing the chance that severe shock to the lamp might dislodge the wick-tube and cause fuel spillage. The wick may be managed by means of common tweezers, e.g. for advancing and trimming the wick without messy fingers.

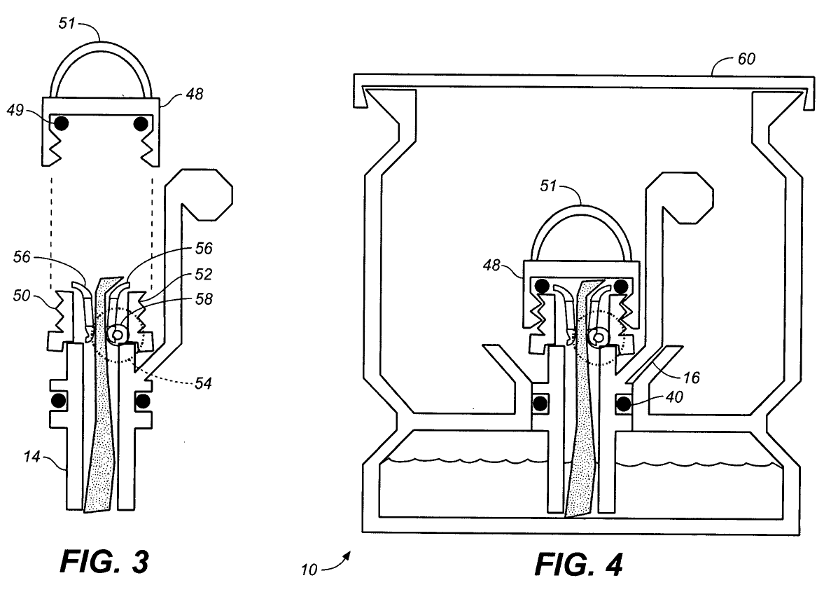

FIG. 3 is a side elevation cross-sectional view of a wick tube assembly. This view shows the wick tube assembly 14, meeting the above specifications for the biodiesel wick tube, equipped with a replaceable cap 48 that seats with a leak-proof seal securely onto either a cap receiving piece 50 (such as fits the screw-on female cap 48 with gasket 49 as depicted) or other articulation on the wick tube itself, and which cap 48 may be equipped with a lanyard 51 to secure one end of a tether connecting the cap to the lamp body to prevent loss.

The wick tube assembly 14 may include a simple wick advance mechanism 52 (such as is commonly found on kerosene lamps) allowing the user to raise or lower the wick with a rotating wick advance control knob 54. Two wick guide plates 56 seated within the top of the wick tube assembly 14 create a horizontally rectangular or oval constriction between them. Within said constriction, a serrated wick advance roller 58 passes through a fitted opening in one of the wick guide plates that function to press the wick against the opposing wick guide plate. The wick advance roller 58 is controlled by the wick advance knob 54 of relatively large diameter mounted on a common wick advance axle. The wick advance knob 54 is of such proportions as are convenient to the fingers of users. Turning the wick advance knob 54 in one direction raises the wick and vice versa, so the wick may be trimmed and fresh wick lit without requiring the user to soil fingers with fuel.

FIG. 4 is a side elevation cross-sectional view of the lamp apparatus of this invention as configured for transport with the wick tube cap in place on the wick tube assembly, and the chimney covered by a chimney cover. The cap 48 may be resealably and securely seated to seal the wick tube assembly 14. The wick tube assembly itself seals the fuel aperture 16 by means of the O-ring 40 around it's circumference fitted to the fuel aperture (or by other means). To prevent loss the cap may also be secured to the lamp by means of a tether secured to a cap lanyard 51 (e.g. when the lamp is in use). Optional chimney cover 60 further isolates the internal volume of the lamp from the outside.

A minimum embodiment of the invention, that is as an oil-candle, omits the affixed chimney but incorporates the novel filling system and specifications for burning vegetable oils as a primary fuel. The absence of a chimney reduces the safety profile of the invention to approximately that of a wax candle in a candle holder. This embodiment realizes a convenient spill and leak-resistant vegetable oil candle, and may be easily modified for use with other liquid fuels, such as kerosene.

The fuel reservoir may be constructed with increased diameter to achieve longer burn times between refills. Due to the constraint on vertical wick height discussed above, the vertical wick height cannot be increased beyond a given value without leaving a puddle of fuel after the flame burns out. The chimney may be expanded as desired beyond the minimum necessary and sufficient specifications described for the preferred embodiment.

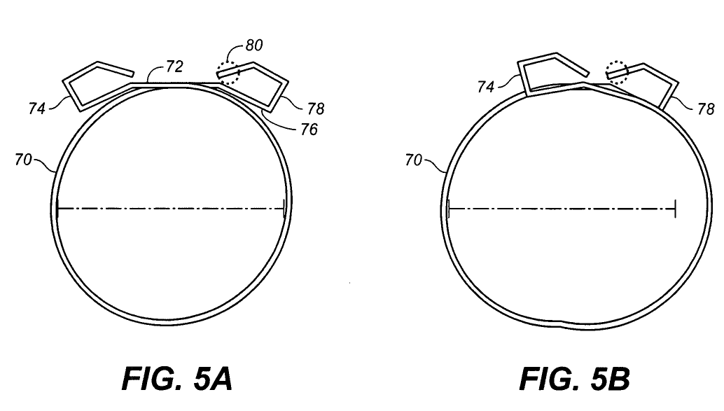

FIGS. 5A and 5B are top plan views of an auxiliary device that allows an accessory to be mounted on the lamp and removed with one hand, and which functions also as a stand for such an accessory that prevents the hot accessory from scorching supporting surfaces. Accessories may be attached at the articulation points provided on the chimney. A flexible steel wire, such as coat hangar wire, that when deformed retains it's new shape and also some flexion, is formed to wrap snugly around the smallest diameter of the outer wall of either or both of the chimney constrictions 30 and 32 to form a base coil 70 of 360 degrees.

The base coil 70 is extended by roughly 30 degrees at one end 72, which extension then turns, roughly perpendicular to the tangent of the coil, away from the chimney to form a finger rest 74, which is a flattish segment, fitted to a human adult index finger, roughly perpendicular to the coil. The base coil then terminates in a loop or any other fashion that suits safety and aesthetics. The coil's other end 76 is extended roughly 30 degrees in the opposite direction, which extension then likewise bends perpendicularly away from the coil to form a second similar but opposing finger rest 78. The coil then extends further as required, usually upward and inward, to form a coupling 80 to hold the accessory concerned in the desired relation to the flame and the lamp generally.

FIG. 5B depicts how the coil is conveniently spread with one hand to release the accessory from the lamp chimney articulations 30 and/or 32. The resulting finger rests 74 and 78 form an angle of approximately 60 degrees to each other, such that a user may easily use the index finger and thumb of one hand to pinch the finger rests toward each other. This causes the base coil 70 of 360 degrees to bend open, increasing it's diameter to exceed that of the lamp bottle/chimney at its largest diameter, allowing the accessory to be lifted away from the lamp with one hand, and similarly replaced. Either constriction 30 and 32 may be used to mount accessories, or both may be used if a particularly secure mounting is desired.

This arrangement allows the base coil 70 of 360 degrees to form a flat horizontal plane that serves as a stand for a properly balanced accessory, allowing the accessory to be rest securely upon any flat surface when detached from the lamp, while any heated parts of the accessory are held well away from that surface.

Accessories that may be attached in this manner include lampshades and high-intensity burst fragrance diffusers. The constrictions may also secure tethers for tote bags for fuel, fragrance, and other small items such as tweezers, squirt fuel bottles, and the like.

The inventive lamp is also suitable as a device to project images of shadow and light and color on nearby surfaces, such as images created by the passage of lamp light through the etched surface of the chimney lamp to project words and pictures on a wall. This function may be enhanced with lenses near, on, or in the walls of the chimney. The lamp is suited for this use by the fact that the flame remains in a fixed position relative to the chimney surface.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting e of the invention, which is defined by the appended claims.

What is claimed as invention is:

1. A liquid fuel lamp apparatus comprising:
a lamp body;
a fuel reservoir in said lamp body, said fuel reservoir having an upwardly opening fuel aperture;
a wick tube assembly for insertion into and sealing engagement with said fuel aperture; and
a wick in said wick tube assembly, wherein when said wick tube assembly is inserted into said fuel aperture, said wick draws fuel from said fuel reservoir through said wick tube assembly to fuel a flame.

2. The liquid fuel lamp apparatus of claim 1 wherein said wick tube assembly includes a bushing for sealing against said fuel aperture.

3. The liquid fuel lamp apparatus of claim 1 wherein said wick tube assembly includes a vertically-extending handle to facilitate insertion and removal of said wick tube assembly from said fuel aperture.

4. The liquid fuel lamp apparatus of claim 1 wherein said fuel aperture comprises a funnel.

5. The liquid fuel lamp apparatus of claim 1 wherein said lamp body includes a fragrance reservoir adjacent said wick tube assembly.

6. The liquid fuel lamp apparatus of claim 1 wherein said lamp body includes a chimney.

7. The liquid fuel lamp apparatus of claim 1 wherein said chimney includes a peripheral constriction for releasable capture of a coil spring accessory holder.

8. The liquid fuel lamp apparatus of claim 1 wherein said chimney includes a removable cover.

9. The liquid fuel lamp apparatus of claim 1 wherein said lamp body includes a peripheral constriction for releasable capture of a coil spring accessory holder.

10. The liquid fuel lamp apparatus of claim 1 wherein said lamp body includes a peripheral constriction to provide a sump for any dripping fuel when said lamp is overturned on its side.

11. The liquid fuel lamp apparatus of claim 1 wherein said wick tube assembly includes a removable sealing cap.

12. The liquid fuel lamp apparatus of claim 1 wherein said wick tube assembly includes a wick advance mechanism.

13. The liquid fuel lamp apparatus of claim 1 wherein said wick tube assembly is constructed of a material of high thermal conductivity and low specific heat so as to become very hot to volatize the fuel in the fuel reservoir when the lamp is lit.

14. The liquid fuel lamp apparatus of claim 1 wherein said wick tube assembly is less than one and one-half inches in height.

15. The liquid fuel lamp apparatus of claim 1 wherein said fuel reservoir contains vegetable oil.
